(19) United States
(12) Patent Application Publication
(10) Pub. No.: US 2025/0146946 A1
(43) Pub. Date: May 8, 2025

(54) DEVICE AND METHOD FOR DETECTION OF ALKALI-SILICA REACTIVITY ON CONCRETE STRUCTURES

(71) Applicant: Board of Trustees of Western Michigan University, Kalamazoo, MI (US)

(72) Inventors: Upul Attanayake, Kalamazoo, MI (US); Naveen Ranasinghe, Kalamazoo, MI (US); Harsha Amunugama, Kalamazoo, MI (US)

(21) Appl. No.: 18/934,582

(22) Filed: Nov. 1, 2024

Related U.S. Application Data
(60) Provisional application No. 63/595,823, filed on Nov. 3, 2023.

(51) Int. Cl. G01N 21/77; G01N 21/88; G01N 33/38

(52) U.S. Cl. CPC G01N 21/77; G01N 21/8851; G01N 2021/7786; G01N 2021/889; G01N 33/383; G01N 2201/06193; G01N 2201/102

(57) ABSTRACT

Embodiments of the disclosure include a remote inspection system for detecting and assessing the alkali-silica reaction (ASR) in situ in concrete, the system including an image acquisition device capable of excluding ambient light from a concrete surface and being placed against and imaging the concrete surface, the image acquisition device comprising a mirrorless camera and daylight and short-range UV light sources wherein the light sources and mirrorless camera are capable of being controlled remotely.

Aspects of the present disclosure includes a method of inspecting in situ the level of ASR present in concrete, including placing an image acquisition device as described above. In yet another aspects, the method further includes acquiring at least one image of said concrete surface prior to treatment with uranyl acetate to assess natural fluorescence in the concrete and acquiring at least one image of said concrete surface after treatment with uranyl acetate.

### CROSS_REFERENCE TO PREVIOUS APPLICATIONS

This application claims the benefit of U.S. Provisional Application No. 63/595,823 filed Nov. 3, 2023, which is incorporated herein in its entirely.

### TECHNICAL FIELD

The present disclosure involves an improved portable device and methods of manufacture and using the same for assessing alkali-silica reactivity of concrete and/or concrete structures in the field using Uranyl Acetate Fluorescent analysis.

### BACKGROUND OF THE INVENTION

Alkali-silica reactivity (ASR) in concrete is one of the major factors affecting its durability, as has been reported in many countries. In fact, ASR has been reported in many structures that have been in service less than a decade. If not properly identified and treated, ASR-affected structures may need to be replaced. ASR, which results from a reaction between cement and the silica found in many aggregates causes the formation of a hygroscopic, soluble and flowable gel of sodium silicate (NaSiO₃·nH₂O) or potassium silicate (KSiO₃·nH₂O), swelling in the presence of moisture and potentially causing cracking, spalling, and concrete failure.

The techniques and procedures currently implemented in the field are in general invasive and/or impractical in many situations. Laboratory techniques require extracting samples (cores) for further testing, which is not permitted for most structures such as highway bridges and nuclear facilities.

The most common in situ procedure is the Uranyl Acetate Fluorescent method, which utilizes the fluorescent properties of uranyl acetate to detect the presence of ASR in concrete. The test involves treating a concrete specimen with uranyl acetate, which can replace the cations in ASR gel with UO²⁺ ions, which produce a characteristic yellowish-green fluorescence under short-wave ultraviolet (UV) light.

The test includes the following steps.

First, the surface of a concrete specimen, (see, e.g., FIG. 7, 701 for a photograph of an example slab) is dampened with distilled water and observed under short-wave UV light to identify the presence of any natural fluorescence. This is called prescreening and helps to avoid later misinterpretation of observations once the surface is treated with uranyl acetate.

Next, the surface is treated with a uranyl acetate solution for a prescribed duration. The solution is prepared by dissolving a specific amount of uranyl acetate powder in an acetic acid solution of a specific concentration.

Then, the treated surface is rinsed with distilled water to remove excess uranyl acetate and immediately observed under daylight and also under a 254 nm short-wave UV light to detect any fluorescent particles.

Finally, the treated sample (see FIG. 7, 702, showing fluorescence of an example slab) shows yellowish-green color (indicated by arrows 703) in the present of ASR, because the UV rays excite the electrons above the ground state and emit photons as electrons transition back to the ground state.

To assess the extent of ASR in the sample, a trained technician/professional needs to look at the color and unique signatures to interpret the data and make conclusions. It is important to note that the interference of ambient light needs to be minimized to collect reliable data. Conventional systems, such as the Spectroline™ UV Darkroom Cabinet with a UV-C (254 nm-365 nm) light, are designed to help to evaluate ASR on typical concrete specimens. (However, the precise wavelength used could be different as listed since the equipment has a UV Long Pass (LP) filter in front of the lamp.) The UV-illuminated fluorescence surfaces can be observed through a contoured eyepiece.

This design lacks several features required for it to be used as an ergonomic, operator-friendly, standalone system, however. The challenges include not having a built-in image sensor to capture a properly exposed image to ultraviolet light, forcing the operator to make decisions based on what is observed through a small, contoured eyepiece; the absence of a dual lighting setup and battery backup; limited portability; and the lack of safety associated with simultaneously holding a bulky apparatus up to a concrete sample in situ while also attempting to detect the fluorescent signals of ASR.

For example, inspections of concrete structures such as bridge beams at an elevation are typical, such that the operator must use a ladder, while holding the inspection device of the prior art. The operator has to stay balanced on a ladder, holding the equipment with both hands, and observing the surface through the eyepiece. Further, the operator needs to find approaches to provide complete darkness over the surface while observing the surface since the opening in the eyepiece is not completely sealed during inspection. With the current device, pictures cannot be taken to document the observations.

Thus, there remains a need for developing a reliable non-invasive methods and procedures to detect ASR in in-service structures. In some embodiments and aspects, the present invention is intended to solve the following limitations of the above-mentioned conventional in situ technology:
(1) Specimen surface is not properly exposed to the UV light because of the fixed position of the built-in light.
(2) Very difficult to place a camera and take sharp images.
(3) Requires a continuous power supply of 120V AC to operate the UV light, a limitation affecting portability.
(4) Interference of ambient light makes it challenging to clearly observe the specimen under the UV light.
(5) Unsafe to assess the condition of a concrete component due to limited workspace and/or access.

With the systems and method of the present invention, one person can hold the unit over the surface while the technician can be at a safe location away from the structure controlling the camera movement, controlling lighting conditions, and observing the surface to analyze the image characteristics to make decision about the condition of the structure. There is no need for an eyepiece, making complete exclusion of ambient light difficult, since the surface image is examined remotely ant the remote controller module. Further, the technician can take and save images for later to include in reports and other documents to describe the condition of the structure.

### BRIEF SUMMARY OF THE INVENTION

One embodiment of the present disclosure includes a remote inspection system for detecting and assessing the alkali-silica reaction (ASR) in concrete, the system including an image acquisition device capable of excluding ambient light from a concrete surface and being placed against and imaging said concrete surface, the image acquisition device comprising a mirrorless camera and daylight and short-range UV light sources wherein said light sources and mirrorless camera are capable of being controlled remotely.

In yet another embodiment, the image acquisition device further comprises a motorized, wireless controlled camera slider, wherein the mirrorless camera is capable of moving along the motorized camera slider to image different parts of the concrete after the image acquisition device is placed on the concrete surface. In some embodiments, the mirrorless camera is a high-definition camera and is capable of being attached to additional lenses or camera mounts. In other embodiments, the bottom of the image acquisition device further includes a gasket capable of molding itself to the concrete surface to block ambient light. In yet other embodiments, the image acquisition device further comprises handles to aid a user in manipulating the device.

Another embodiment of the system may further include a remote controller device capable of operating said mirrorless camera and said daylight and short-range UV light sources. In other embodiments, the remote controller device further includes a display capable of viewing images and/or controlling said mirrorless camera and day and UV lights.

In yet other embodiments of the system, the remote controller and image acquisition device are capable of each being used by different users during analysis of concrete surfaces.

In other embodiments of the system, the system further includes a post-processing system capable of helping to diagnose the level of ASR present in an image of said concrete surface.

One aspect of the present disclosure includes a method of inspecting in situ the level of ASR present in concrete, including placing an image acquisition device capable of excluding ambient light on the surface of said concrete, the image acquisition device including a mirrorless camera and daylight and short-range UV light sources wherein said light sources and mirrorless camera are capable of being controlled remotely.

In yet another aspects, the method further includes acquiring at least one image of said concrete surface prior to treatment with uranyl acetate to assess natural fluorescence in the concrete and acquiring at least one image of said concrete surface after treatment with uranyl acetate.

In yet other aspects of a method of the present disclosure, the image acquisition device further includes a radio receiver that is capable of receiving communications to control said mirrorless camera and light sources. In other aspects, the image acquisition device further includes a motorized, wireless controlled camera slider, wherein said mirrorless camera is capable of moving along said motorized camera slider to image different parts of the concrete after said image acquisition device is placed on the concrete surface. In others, the mirrorless camera is a high-definition camera and is capable of being attached to additional lenses or camera mounts. In others, the bottom of the image acquisition device further comprises a gasket capable of molding itself to the concrete surface to block ambient light.

In yet other aspects of the method, the image acquisition device is capable of being controlled by a remote controller device to control said mirrorless camera and light sources. In others, the remote controller device further comprises a display capable of viewing images and/or controlling said mirrorless camera and day and UV lights.

In yet other aspects of a method of the present disclosure, the remote controller and image acquisition device are capable of each being used by different users during analysis of concrete surfaces.

In other aspects, the method further includes analyzing said images for the presence of the color signatures of ASR. In yet other aspects of methods of the present disclosure, analyzing said images for the presence of the color signatures of ASR further includes using a post-processing system capable of helping to diagnose the level of ASR present in an image of said concrete surface.

### BRIEF DESCRIPTION OF THE DRAWINGS

The present invention(s) are illustrated by way of example and not limitation with reference to the accompanying drawings, in which like references generally indicate similar elements or features.

FIG. 1 illustrates a system diagram of an ASR inspection device according to an example embodiment of the present disclosure;

FIG. 2 illustrates an isometric view of an example embodiment of an ASR inspection device according to the present disclosure;

FIG. 3 illustrates a top view of an example embodiment of an ASR inspection device according to the present disclosure;

FIG. 4 illustrates a bottom view of an example embodiment of ASR inspection device according to the present disclosure;

FIG. 5 illustrates a controller of ASR inspection device according to an example embodiment of the present disclosure;

FIG. 6 illustrates a camera mounted slider of ASR inspection device according to an example embodiment of the present disclosure;

FIG. 7 illustrates a concrete sample and a uranyl acetate treated specimen observed under different lighting conditions of ASR inspection for some embodiments and aspects of the present disclosure;

FIG. 8 depicts the control system for operating the sensor remotely in some embodiments and aspects of the present disclosure;

FIG. 9 depicts an image acquisition module used in some aspects and embodiments of the present disclosure.

In general, element with the same number as for the same element in different figures for clarity. However, this does not necessarily imply that the same example embodiment or aspect is represented in the various drawings herein, and other elements and embodiments and aspects or combinations thereof will be readily apparent to persons of skill in the art.

### DETAILED DESCRIPTION

Various embodiments of the invention are described more hereafter with reference to accompanying drawings, in which some, but not all embodiments are shown in the figures.

Throughout the specification, references made to “top” and “bottom” of the device or other parts of the device in relation to each other are used for descriptive purposes only and refer to the situation where the device is placed on top of a horizontal concrete surface for testing and viewing from above the slab. One advantage of the device, in some embodiments, is its portability to image concrete surfaces in any orientation (from below (“upside down”) such to test the underside of a bridge span; vertical surfaces, etc.), thus the directions used for description are not intended to limit the device only to the use case of being placed above horizontal surfaces, and persons of skill in the art will recognize others are possible and intended with embodiments of the portable device presented herein.

This present disclosure relates to a novel inspection device, support method and firmware for identifying concrete having deleterious alkali-silica reaction (ASR) gels, Embodiments and aspects of present disclosure allow detection of ASR-affected areas precisely with correct lighting either under field or laboratory conditions. The present disclosure may consist, in some aspects and embodiments, of an image acquisition device; remote controller, which may in some embodiments, be separate from the image acquisition device and control it remotely; and the post-processing system in some embodiments.

In some embodiments and aspects, the image acquisition device may include a mirrorless camera, short-wave UV lights and e day lights (i.e., fluorescent lights or others that produce a spectrum similar to daylight) to observe the specimens, a motorized camera slider, a wireless control system, and a battery compartment. The image acquisition device remote-control system of aspects and embodiments of the present invention removes the need to have room to position, hold and monitor testing results using a bulky device, allowing the operator to remain away from the structure to carefully observe the surface and capture necessary images while another person holds the equipment over the surface being inspected. This allows for faster and easier inspections and multiple and hard-to-access points.

In some embodiments, a post processing system consists of an artificial intelligence (AI) model that analyses and recognizes color signatures of ASR. The identified signatures are stored in a cloud database to be utilized in future detection processes. Furthermore, this intelligent inspection device is implemented to overcome the existing field inspection challenges with state-of-the-art technology.

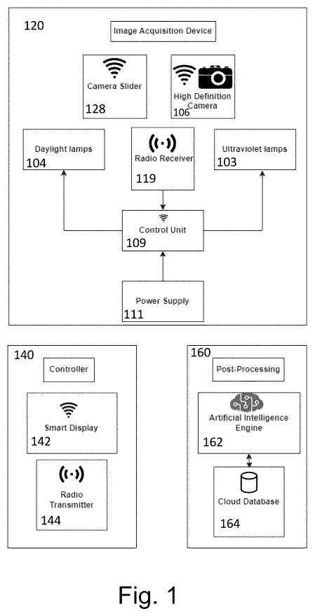

FIG. 1 depicts a diagram of three modules of the present disclosure, in some aspects and embodiments, comprising an image acquisition device 120, controller 140 and post-processing module 160. Images are acquired, in some aspects and embodiments by a wirelessly controllable high definition camera 106, which is capable of being moved on a wireless controllably camera slider 128. The image acquisition device 120 can communicate via radio receiver 119 to controller 140; such communication may be via 2.4 Ghz radio antenna 110, wireless, electric signals via wire, or any means known in the art). Control unit 109 may control daylight lamps 104 and UV lamps 103 and be powered by an onboard power supply 111 (i.e., a battery in some embodiments). Control unit 109 may also communicate with camera slider 128 or camera 106 upon receiving signals from controller 140, or controller 140 may send signals to these devices 128, 106 directly.

Still referring to FIG. 1, In some aspects and embodiments, Controller 140 consists of a smart display 142 capable of viewing images sent from device 120, controlling device 120 and components thereon. Communication with device 120 may be facilitated with radio controller 144 in some aspects and embodiments.

Still referring to FIG. 1, in some aspects and embodiments, post-processing system 160 (located on a stand-alone device or directly on controller 140, or device 120. In some embodiments, artificial intelligence engine 162 may communicate with a cloud database 164 to diagnose the level of ASR in visuals of concrete samples acquired by 120, 140. In some aspects and embodiments, the color and grayscale images may be processed with RGB, HSV and CMYK color spaces. In some aspects or embodiments, artificial intelligence and machine learning models may be used to enhance ASR detection.

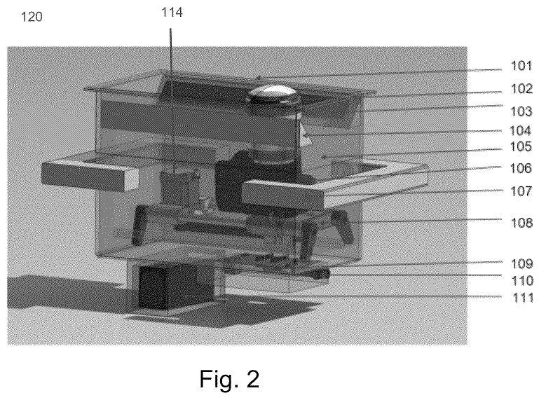

FIG. 2 illustrates one example embodiment of the image acquisition device 120 of the present disclosure. The device 120 may be made of any suitable, opaque and sturdy material, preferably lightweight to facilitate use of the device in situ. In some embodiments, the enclosure may be made with Polycarbonate (PC) filament to provide the required strength, impact resistance, and stiffness. In some embodiments, the bottom of the enclosure comprises a foam (or other suitable material) gasket 101 to prevent external light entering into the chamber. The device may further consist of a mirrorless camera 105 to capture images under daylight-equivalent (i.e., fluorescent tube, LED light or the like) 104 and UV light 103, as well as various lenses 102 and extended mounts 105 to be used in different scenarios such as laboratory and field conditions.

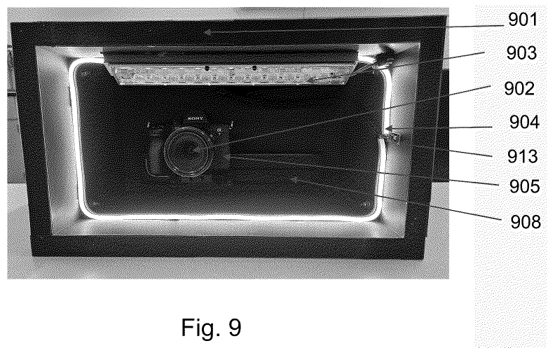

Handles 107 are used to firmly hold down the device 120 to the surface to be examined. The camera 106 is mounted on a slider 108 that allows remotely or manually moving the camera over the desired position to capture images as shown below in FIG. 9. The controller 109 is used to operate the system remotely, such as operating, moving and adjusting the camera and the lighting system. Motor 114 is controlled by controller 109 and can pull camera along the sliders 108.

The controller optionally has a display, e.g., a smartphone 110 to monitor the camera position directly, and/or send input signals to the motorized slider and the mirrorless camera to position the camera over the desired location and capture images wirelessly. Finally, a rechargeable battery 111 (such as a Lithium-Polymer rechargeable battery) supplies power in some embodiments, making this a standalone device.

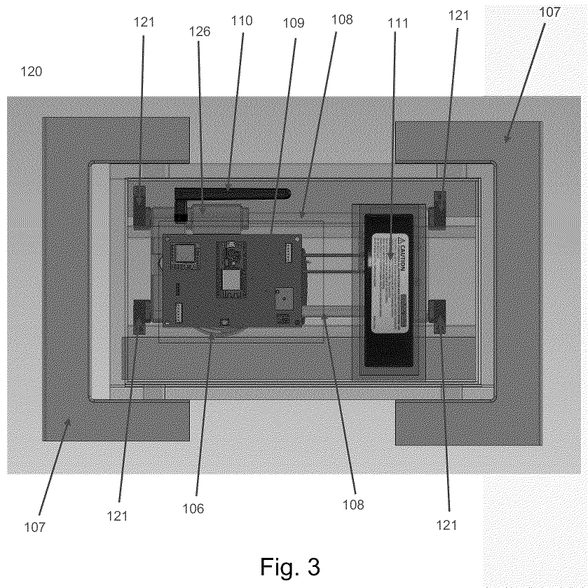

FIG. 3 depicts the top of example embodiment 120 shown in FIG. 1. Although other placements are possible, in this embodiment, rechargeable battery 111 is located above one or more slider(s) 108 and controller 109 for easy changing out of the battery 111 as needed. Slider 108 is held in place by feet 121, which allows controller 109 to move camera 106 along slider(s) 108 within 120 as needed to adjust the view for the operator.

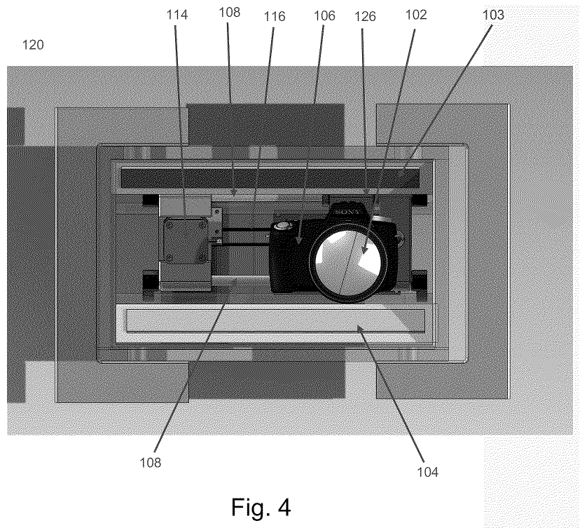

FIG. 4 depicts the bottom of example embodiment 120, with camera 106, equipped with lens 102, and shows UV light 103 and daylight 104 to view concrete samples under varying light conditions. Motor 114 is shown with belt 116, which allows the camera 106 to be automatically moved laterally along sliders 108 as needed for sample viewing.

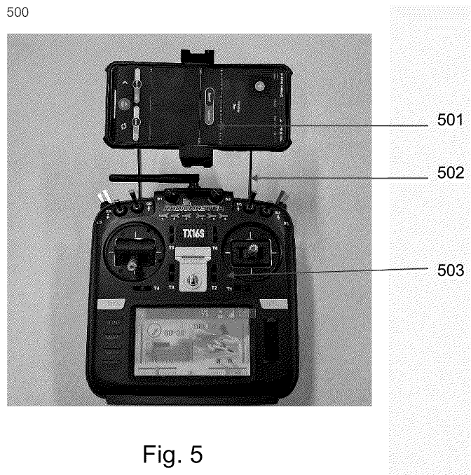

FIG. 5 depicts an example embodiment 500 of a remote controller of the present invention. This particular embodiment uses the Radio Master TS16S, a high-end radio transmitter 503 that features an advanced multi-protocol system with 16 data channels. This allows the remote operation of the system and precise and accurate control of UV and daylight settings. The transmitter also features a high-speed communication system that ensures fast and reliable transmission of data between the transmitter and receiver.

Smartphone 501 mounted on and attached to transmitter 501 via suitable mounting means 502. In some embodiments, this allows controlling the precise movements of the camera slider and the image-capturing process. The recorded images may be saved in a camera storage device and/or on the smartphone 501 and can be retrieved later for further analysis and reporting.

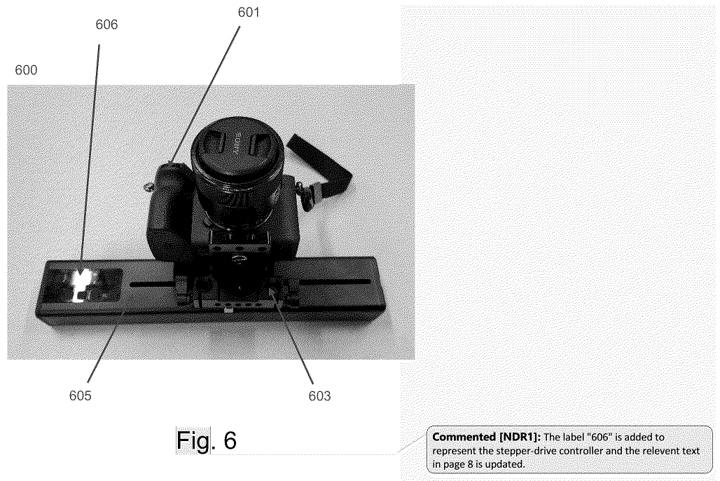

FIG. 6 shows an example embodiment of the present disclosure. Here, a Sony a-IV camera is shown, which is a powerful Exmor-R 33 Megapixel image sensor designed to capture high-resolution photos and videos; one of its notable features is its ability to capture ultraviolet fluorescence images. This is made possible by its advanced sensor technology and the integration of specialized filters. Ultraviolet fluorescence imaging is a technique used to capture images of fluorescent materials that emit light in response to UV radiation. The specialized filters allow detecting UV light in the 280-400 nm range. The use of filters enables the camera to capture ultraviolet fluorescence at high sensitivity (i.e., 50-204,800 ISO) and resolution (7008×4672 pixels). In addition, the advanced sensor technology in the camera reduces noise and improves image quality. The image stabilization capability and portability make this camera an ideal choice for fieldwork and research applications.

As known in the art, any suitable camera or image-capturing device may be used capable of taking, storing and/or transmitting photos, especially those in the UV range, to a storage and/or display device may be used as well.

The camera 601 is mounted via mount 603 to a motorized slider 605 with wireless connectivity and uses precision bearings to ensure smooth and precise movement of the camera. The stepper-drive system in the slider 605 allows for quiet and smooth operation while the brake system allows for precise positioning and locking of the camera at any point during the movement. The controller 606 allows to adjust the speed of the stepper-drive system.

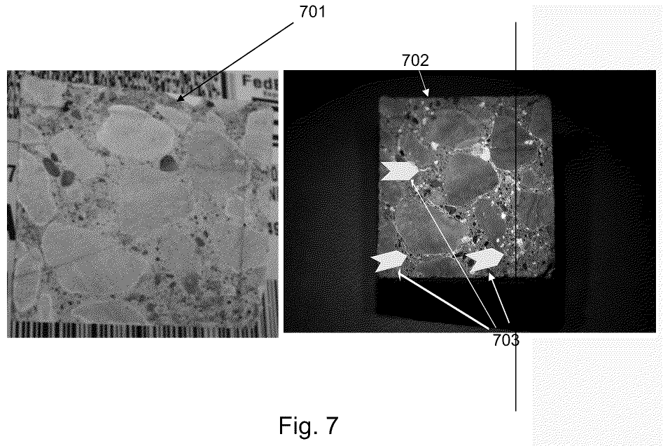

FIG. 7 depicts example concrete images taken with an embodiment of the present invention. A concrete sample 701 is observed under daylight for reference. Under UV light 702, the treated sample shows yellowish-green color (indicated by arrows 703) in the present of ASR, because the UV rays excite the electrons above the ground state and emit photons as electrons transition back to the ground state.

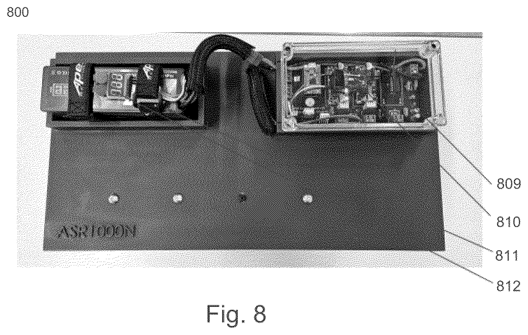

FIG. 8 shows an example embodiment 800 of remote controller (i.e., FIG. 1, 140) of the present disclosure. Controller 800 is used to operate the system remotely, such as operating the camera and the lighting system. The controller has a display, e.g., a smartphone 810 to send input signals to the motorized slider and the mirrorless camera to position the camera over the desired location and capture images wirelessly. Finally, a rechargeable battery 811 (such as a Lithium-Polymer rechargeable battery) supplies power making this a standalone device. The battery voltage monitor 812 is employed to observe the battery voltage and provide an early warning for a low voltage situation.

FIG. 9 depicts an example embodiment of the image acquisition device 900 with the camera 905 and slider 908 shown in FIG. 8 mounted. Device 900 has a high-density foam gasket 901 to prevent the external light entering the chamber. Device 900 includes various lenses 902 and extended mounts 905 to be used in different scenarios such as laboratory and field conditions. UV light 903 and daylight sources 904 are also included. In some embodiments, a UV light sensor 913 prevents the operator being accidentally exposed to UV radiation.

Some aspects and embodiments of the present disclosure include an inspection device, support method, and firmware for identifying concrete containing gels formed by the alkali-silica reaction (ASR) comprising: assessing the concrete structure using an image sensor with image processing capabilities; and comprising a wireless movable motorized platform, mirrorless camera controlled using a remote controller; and daylight and short-range Ultraviolet-C lights (254 nm) to illuminate the surface; and including extended mounts and various lens to use in laboratory conditions and different field settings; and detecting hits associated with cracking and expansion of the concrete structure resulting from the alkali-silica reaction using an image sensor and image processing; and evaluating the cracking and expansion of the concrete structure resulting from the alkali-silica reaction by classifying the affected areas.

In some embodiments and aspects, the method and device can detect micro-cracks, map-cracks, and/or longitudinal cracks.

In some aspects and embodiments, the method and device can detect alkali-silica reactions forming via Na₂SiO₃ and/or K₂SiO₃. In some aspects and embodiments, the method and device are used to assess the concrete structure's cracking and expansion.

In some aspects and embodiments, the method and device may process color and/or grayscale images obtained from the image acquisition device with RGB, HSV and CMYK color spaces. In others, artificial intelligence and machine learning models may be used to enhance ASR detection.

### Algorithm

The presented algorithm provides an example script used to control the lighting system

```
  /*
    ASR1000N Main Script
    This program allows to control the light conditions inside the
    chamber using 2.4GHz
    remote link.
  */
  int sensorPin = A0;
  int sensorValue = 0;
  int sensorPin2 = A1;
  int sensorValue1 = 0;
  void setup( ) {
    Serial.begin(115200);
    pinMode(sensorPin, INPUT);
    pinMode(sensorPin, INPUT);
  }
  void loop( ) {
    sensorValue = pulseIn(sensorPin, HIGH);
    Serial.print(sensorValue);
  if (sensorValue < 1000) {
    Serial.printIn(“A”);
    analogWrite(10, 0);
    analogWrite(11, 0);
    goto test;
  } else if (1901 < sensorValue) {
    Serial.printIn(“C”);
    analogWrite(11, 125);
    analogWrite(10, 0);
    goto test;
  } else if (1200 < sensorValue < 1800) {
    Serial.printIn(“B”);
    analogWrite(10, 125);
    analogWrite(11, 0);
    goto test;
  }
test:
  Serial.printIn(“measurement completed”);
}
```

1. A remote inspection system for detecting and assessing the alkali-silica reaction (ASR) in concrete, comprising:
an image acquisition device capable of excluding ambient light from a concrete surface and being placed against and imaging said concrete surface,
comprising
a mirrorless camera, and
daylight and short-range UV light sources
wherein said light sources and mirrorless camera are capable of being controlled remotely.

2. The remote inspection system of claim 1, further comprising:
a remote controller device capable of operating said mirrorless camera and said daylight and short-range UV light sources.

3. The remote inspection system of claim 1, wherein the image acquisition device further comprises
a motorized, wireless controlled camera slider,
wherein said mirrorless camera is capable of moving along said motorized camera slider to image different parts of the concrete after said image acquisition device is placed on the concrete surface.

4. The remote inspection system of claim 1, wherein said mirrorless camera is a high-definition camera and is capable of being attached to additional lenses or camera mounts.

5. The remote inspection system of claim 1, wherein the bottom of the image acquisition device further comprises a gasket capable of molding itself to the concrete surface to block ambient light.

6. The remote inspection system of claim 1, wherein the image acquisition device further comprises
handles to aid a user in manipulating the device.

7. The remote inspection system of claim 2, wherein the remote controller device further comprises
a display capable of viewing images and/or controlling said mirrorless camera and day and UV lights.

8. The remote inspection system of claim 7, wherein the remote controller and image acquisition device are capable of each being used by different users during analysis of concrete surfaces.

9. The remote inspection system of claim 1 further comprising:
a post-processing system capable of helping to diagnose the level of ASR present in an image of said concrete surface.

10. A method of inspecting in situ the level of ASR present in concrete, comprising:
placing an image acquisition device capable of excluding ambient light on the surface of said concrete, said image acquisition device comprising
a mirrorless camera and
daylight and short-range UV light sources
wherein said light sources and mirrorless camera are capable of being controlled remotely.

11. The method of claim 10 further comprising:
acquiring at least one image of said concrete surface prior to treatment with uranyl acetate to assess natural fluorescence in the concrete, and
acquiring at least one image of said concrete surface after treatment with uranyl acetate.

12. The method of claim 10 further comprising:
analyzing said images for the presence of the color signatures of ASR.

13. The method of claim 10, wherein said image acquisition device further comprises
a radio receiver that is capable of receiving communications to control said mirrorless camera and light sources.

14. The method of claim 10, further comprising a remote controller device capable of controlling said mirrorless camera and light sources.

15. The method of claim 10, wherein the image acquisition device further comprises
a motorized, wireless controlled camera slider, wherein said mirrorless camera is capable of moving along said motorized camera slider to image different parts of the concrete after said image acquisition device is placed on the concrete surface.

16. The method of claim 10, wherein the mirrorless camera is a high-definition camera and is capable of being attached to additional lenses or camera mounts.

17. The method of claim 10, wherein the bottom of the image acquisition device further comprises
a gasket capable of molding itself to the concrete surface to block ambient light.

18. The method of claim 14, wherein said remote controller device further comprises
a display capable of viewing images and/or controlling said mirrorless camera and day and UV lights.

19. The method of claim 14, wherein the remote controller and image acquisition device are capable of each being used by different users during analysis of concrete surfaces.

20. The method of claim 12, wherein said analyzing said images for the presence of the color signatures of ASR uses a post-processing system capable of helping to diagnose the level of ASR present in an image of said concrete surface.
